(19) United States
(12) Patent Application Publication
(10) Pub. No.: US 2026/0250294 A1
(43) Pub. Date: Aug. 27, 2026

(54) Preparation Of A Pyrimidinyl-3,8-Diazabicyclo[3.2.1]Octanylmethanone Derivative And Salt Thereof

(71) Applicant: PFIZER INC., New York, NY (US)

(72) Inventors: Adam Edward GOETZ, New York, NY (US); Sally Gut RUGGERI, New York, NY (US); Robert Alan SINGER, New York, NY (US)

(21) Appl. No.: 19/652,160

(22) Filed: Apr. 20, 2026

Related U.S. Application Data
(62) Division of application No. 18/004,109, filed on Jun. 30, 2021.
(60) Provisional application No. 63/047,590, filed on Jul. 2, 2020.

(51) Int. Cl. C07D 487/08

(52) U.S. Cl. CPC C07D 487/08

(57) ABSTRACT

Methods for preparing ((S)-2,2-difluorocyclopropyl)-((1R,5S)-3-(2-((1-methyl-1H-pyrazol-4-yl)amino)pyrimidin-4-yl)-3,8-diazabicyclo[3.2.1]-octan-8-yl)methanone and intermediates used in the processes of preparation thereof.

### CROSS-REFERENCE TO RELATED APPLICATIONS

This application is a divisional application of U.S. application Ser. No. 18/004,109, filed on Jan. 3, 2023, which is a national stage application of PCT/IB2021/055854, filed on Jun. 30, 2021, which claims the benefit of U.S. Provisional Application No. 63/047,590, filed Jul. 2, 2020, the contents of each of which is herein incorporated by reference in its entirety.

### FIELD OF THE INVENTION

The present invention relates to methods for preparing ((S)-2,2-difluorocyclopropyl)-((1R,5S)-3-(2-((1-methyl-1H-pyrazol-4-yl)amino)pyrimidin-4-yl)-3,8-diazabicyclo[3.2.1]-octan-8-yl)methanone, a compound useful for inhibiting Janus Kinases (JAKs). The invention also relates to intermediates for preparing said compound.

### BACKGROUND OF THE INVENTION

((S)-2,2-Difluorocyclopropyl)-((1R,5S)-3-(2-((1-methyl-1H-pyrazol-4-yl)amino)-pyrimidin-4-yl)-3,8-diazabicyclo[3.2.1]octan-8-yl)methanone has the chemical formula C₁₈H₂₁F₂N₇O and the following structural formula:

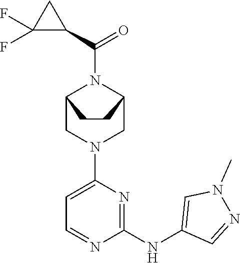 [SMILES: Cn1cc(Nc2nccc(N3C[C@H]4CC[C@@H](C3)N4C(=O)[C@@H]3CC3(F)F)n2)cn1]

A prior synthesis of ((S)-2,2-difluorocyclopropyl)-((1R,5S)-3-(2-((1-methyl-1H-pyrazol-4-yl)amino)-pyrimidin-4-yl)-3,8-diazabicyclo[3.2.1]octan-8-yl)methanone is described in commonly assigned U.S. Pat. No. 9,663,526, the contents of which are incorporated herein by reference in its entirety. The crystalline form of ((S)-2,2-difluorocyclopropyl)-((1R,5S)-3-(2-((1-methyl-1H-pyrazol-4-yl)amino)-pyrimid-in-4-yl)-3,8-diazabicyclo[3.2.1]-octan-8-yl)methanone free base, is useful as an inhibitor of protein kinases, such as the enzyme Janus Kinase and as such is useful therapeutically as an immunosuppressive agent for organ transplants, xenotransplantation, lupus, multiple sclerosis, rheumatoid arthritis, psoriatic arthritis, inflammatory bowel disease (IBD), psoriasis, Type I diabetes and complications from diabetes, cancer, asthma, atopic dermatitis, autoimmune thyroid disorders, ulcerative colitis, Crohn's disease, Alzheimer's disease, Leukemia and other indications where immunosuppression would be desirable.

Accordingly, it is desirable to provide more efficient methods of manufacturing ((S)-2,2-difluorocyclopropyl)-((1R,5S)-3-(2-((1-methyl-1H-pyrazol-4-yl)amino)-pyrimidin-4-yl)-3,8-diazabicyclo[3.2.1]octan-8-yl)methanone and its p-toluenesulfonic acid salt, affording the product in higher yield and superior purity.

### SUMMARY OF THE INVENTION

The present invention provides a method for preparing a compound of formula I:

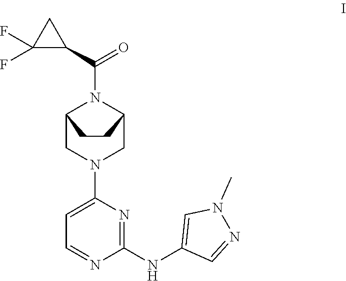 [SMILES: Cn1cc(Nc2nccc(N3C[C@H]4CC[C@@H](C3)N4C(=O)[C@@H]3CC3(F)F)n2)cn1]

comprising (a) (i) preparing a salt from a compound having the structure:

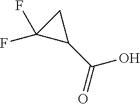

and a base having the structure:

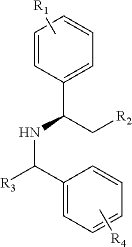

wherein R₁, R₂, R₃, and R₄ are each independently selected from the group consisting of hydrogen, halo, hydroxy, C₁-C₆ alkyl and C₁-C₆ alkoxy; or,
(ii) preparing an activated ester having the structure:

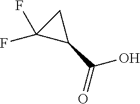

wherein R is selected from the group consisting of C₆-C₁₂ aryl and C₄-C₉ heteroaryl, wherein said C₆-C₁₂ aryl and C₄-C₉ heteroaryl are optionally substituted with a C₁-C₆ alkyl, —S(═O)—R₀, —S(═O)₂—R₀, cyano, nitro, C₁-C₆ alkoxy, or halo, where R₀ is C₁-C₆ alkyl;
(b) reacting said activated ester or said salt with a compound having the structure:

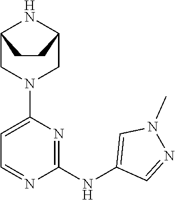 [SMILES: Cn1cc(Nc2nccc(N3C[C@H]4CC[C@@H](C3)N4)n2)cn1]

under conditions suitable to form the compound of formula I.

The present invention will be further understood from the following description given by way of example only. The present invention is directed to methods for the preparation of ((S)-2,2-difluorocyclopropyl)-((1R,5S)-3-(2-((1-methyl-1H-pyrazol-4-yl)amino)pyrimidin-4-yl)-3,8-diazabicyclo[3.2.1]-octan-8-yl)methanone and novel intermediates thereto. While the present invention is not so limited, an appreciation of various aspects of the invention will be gained through the following discussion and the examples.

The term “alkyl,” as used herein, means a straight or branched chain monovalent hydrocarbon group of formula —CₙH₍₂ₙ₊₁₎. Non-limiting examples include methyl, ethyl, propyl, butyl, 2-methyl-propyl, 1,1-dimethylethyl, pentyl and hexyl.

The term “alkoxy,” as used herein, means an alkyl substituent attached through an oxygen atom. Non-limiting examples include methoxy, ethoxy, propoxy, butoxy, pentoxy, and hexyloxy.

The term “benzyl,” as used herein, means a phenylmethyl group.

The term “aryl,” as used herein, means a 6 to 8-membered monocyclic or 6 to 12-membered bicyclic carbocycle which is aromatic or partially unsaturated, said carbocycle being optionally substituted by one or more groups R. Examples include phenyl or naphthalenyl.

The term “heteroaryl,” as used herein, refers to a monocyclic or bicyclic aromatic hydrocarbon containing from 5 to 10 ring atoms in which at least one of the ring carbon atoms has been replaced with a heteroatom selected from oxygen, nitrogen and sulfur. Such a heteroaryl group may be attached through a ring carbon atom or, where valency permits, through a ring nitrogen atom. Common examples of 10-membered heteroaryl groups include quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl and pyrimido[4,5-d]pyrimidinyl.

The term “halogen,” or “halo,” as used herein, refers to fluoride, chloride, bromide, or iodide.

The term “amino,” as used herein, refers to —NH₂.

When a substituent is defined as a combination of two groups (e.g., alkoxyalkyl) the moiety concerned is always attached through the second of the two groups named (in this case alkyl). Thus, for example, ethoxymethyl corresponds to CH₃CH₂—O—CH₂—.

Unless otherwise defined herein, scientific and technical terms used in connection with the present invention have the meanings that are commonly understood by those of ordinary skill in the art.

If substituents are described as being “independently selected” from a group, each substituent is selected independent of the other. Each substituent therefore may be identical to or different from the other substituent(s).

### BRIEF DESCRIPTION OF THE DRAWINGS

FIG. 1 provides a powder X-ray diffraction pattern obtained for crystalline 2,2-difluorocyclopropane-1-(S)-carboxylate (R)—N-benzyl-1-phenylethan-1-aminium salt as set forth in Preparation 3 hereinbelow.

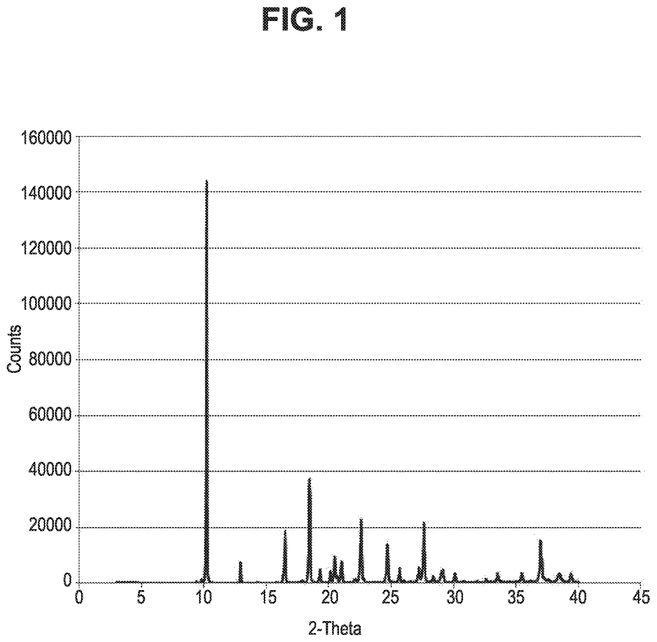

FIG. 2 provides a powder X-ray diffraction pattern obtained for crystalline bis[(1R,5S)-8-benzyl-3,8-diazabicyclo[3.2.1]octane][1,1′-biphenyl]-4,4′-diol complex having the structure:

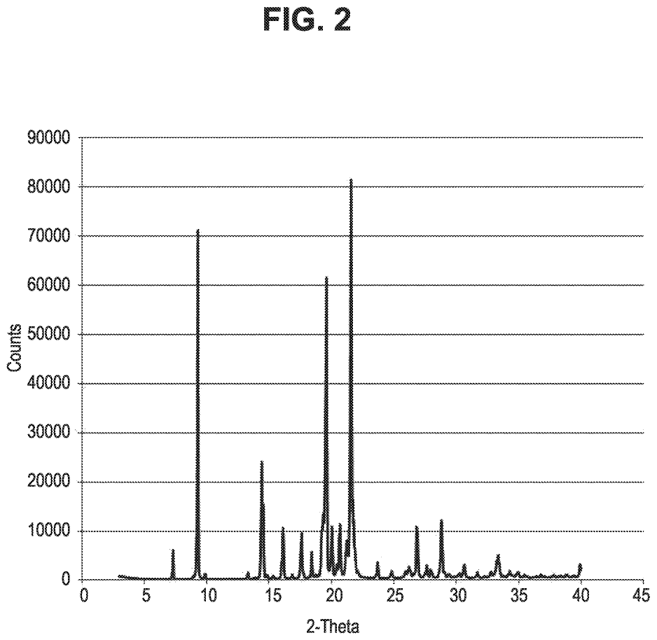

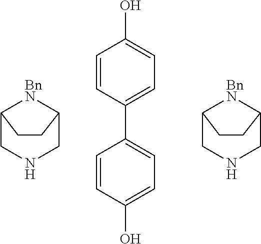

FIG. 3 provides a powder X-ray diffraction pattern obtained for crystalline (1R,5S)-8-benzyl-3,8-diazabicyclo[3.2.1]octane as set forth in Preparation 1 hereinbelow.

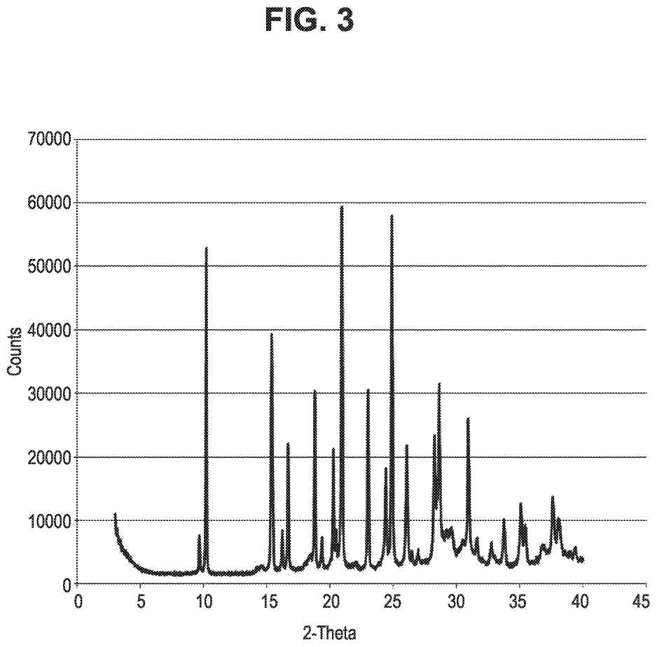

### DETAILED DESCRIPTION OF THE INVENTION

According to a first aspect of the invention, there is provided a method for preparing a compound of formula I:

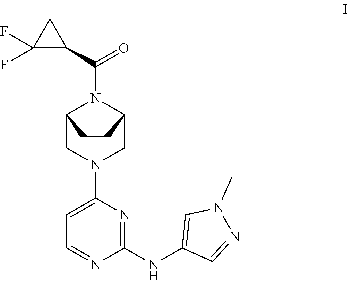 [SMILES: Cn1cc(Nc2nccc(N3C[C@H]4CC[C@@H](C3)N4C(=O)[C@@H]3CC3(F)F)n2)cn1]

comprising (a) (i) preparing a salt from a compound having the structure:

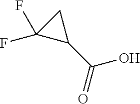

and a base having the structure:

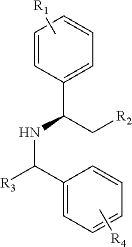

wherein R₁, R₂, R₃, and R₄ are each independently selected from the group consisting of hydrogen, halo, hydroxy, C₁-C₆ alkyl and C₁-C₆ alkoxy; or,
(ii) preparing an activated ester having the structure:

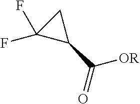

wherein R is selected from the group consisting of C₆-C₁₂ aryl and C₄-C₉ heteroaryl, wherein said C₆-C₁₂ aryl and C₄-C₉ heteroaryl are optionally substituted with a C₁-C₆ alkyl, —S(═O)—R₀, —S(═O)₂—R₀, cyano, nitro, C₁-C₆ alkoxy, or halo, where R₀ is C₁-C₆ alkyl;
(b) reacting said activated ester or said salt with a compound of formula IV:

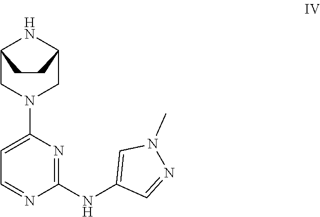 [SMILES: Cn1cc(Nc2nccc(N3C[C@H]4CC[C@@H](C3)N4)n2)cn1]

under conditions suitable to form the compound of formula I.

Described below are a number of embodiments (E) of this first aspect of the invention, where for convenience E1 is identical thereto.

E1. The method of preparing a compound of formula I, as defined above.

E2. The method of E1, wherein R₁, R₂, R₃, and R₄ are hydrogen.

E3. The method of E1 or E2, wherein R isp-cyanophenyl or isoquinolin-3-yl.

E4. A method for preparing the p-toluenesulfonic acid salt of a compound of formula I:

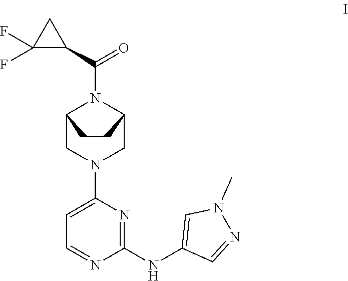 [SMILES: Cn1cc(Nc2nccc(N3C[C@H]4CC[C@@H](C3)N4C(=O)[C@@H]3CC3(F)F)n2)cn1]

comprising (a) (i) preparing a salt from a compound having the structure:

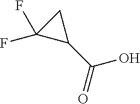

and a base having the structure:

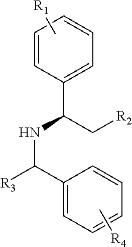

wherein R₁, R₂, R₃, and R₄ are each independently selected from the group consisting of hydrogen, halo, hydroxy, C₁-C₆ alkyl and C₁-C₆ alkoxy; or,
(ii) preparing an activated ester having the structure:

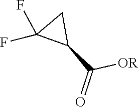

wherein R is selected from the group consisting of C₆-C₁₂ aryl and C₄-C₉ heteroaryl, wherein said C₆-C₁₂ aryl and C₄-C₉ heteroaryl are optionally substituted with a cyano, —S(═O)—R₀, —S(═O)₂—R₀, nitro, C₁-C₆ alkoxy, or halo, where R₀ is C₁-C₆ alkyl;
(b) reacting said activated ester or said salt with a compound of formula IV:

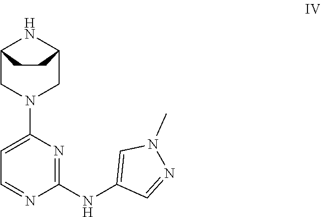 [SMILES: Cn1cc(Nc2nccc(N3C[C@H]4CC[C@@H](C3)N4)n2)cn1]

under suitable conditions to form the compound of formula I:

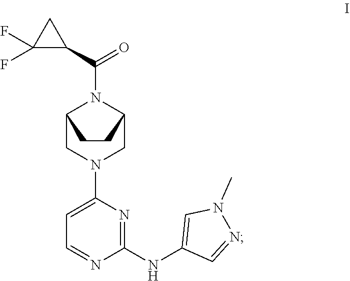 [SMILES: Cn1cc(Nc2nccc(N3C[C@H]4CC[C@@H](C3)N4C(=O)[C@@H]3CC3(F)F)n2)cn1]

(c) treating said compound with p-toluenesulfonic acid under suitable conditions to afford the p-toluenesulfonic acid salt of the formula IA:

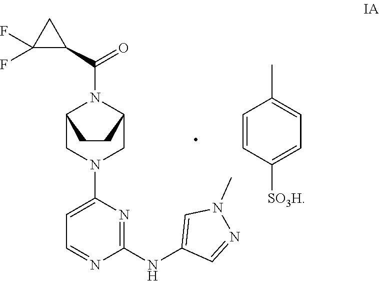

E5. The method of E4, wherein R₁, R₂, R₃, and R₄ are hydrogen.

E6. The method ofany one of E4 or E5, wherein R is p-cyanophenyl or isoquinolin-3-yl.

E7. A method for preparing a salt of formula II:

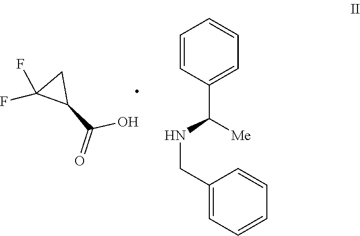 [SMILES: O=C(O)[C@@H]1CC1(F)F ; C[C@@H](NCc1ccccc1)c1ccccc1]

comprising (a) preparing a carboxylic acid having the structure:

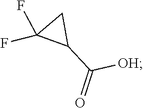

and,
(b) reacting said carboxylic acid with a compound having the structure:

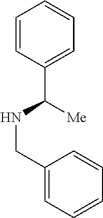 [SMILES: C[C@@H](NCc1ccccc1)c1ccccc1]

under suitable conditions to form the salt of formula II.

E8. The method of E7, wherein the carboxylic acid is prepared by treating a compound having the structure:

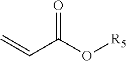

wherein R₅ is C₁-C₆ alkyl, C₃-C₆ cycloalkyl, benzyl, C₆-C₁₂ aryl, or C₄-C₉ heteroaryl with an ester compound having the structure:

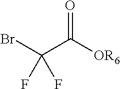

wherein R₆ is C₁-C₅ alkyl, benzyl, C₆-C₁₂ aryl, or C₄-C₉ heteroaryl.

E9. The method of E8 wherein R₅ is n-propyl or n-butyl, and R₆ is methyl or ethyl.

E10. The method according to any one of E7 to E9, wherein the reaction is carried out using n-Bu₄NBr, n-Bu₄NI or n-Bu₄NOH.

E11. The method of E7, wherein the carboxylic acid is prepared by (a) treating a compound having the structure:

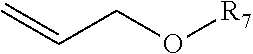

wherein R₇ is C₂-C₆ alkyl, C₃-C₆ cycloalkyl, benzyl, C₆-C₁₂ aryl, and C₄-C₉ heteroaryl with an ester compound having the structure:

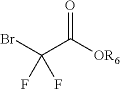

wherein R₆ is C₁-C₅ alkyl, benzyl, C₆-C₁₂ aryl, or C₄-C₉ heteroaryl under suitable conditions to form an intermediate having the structure:

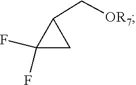

(b) treating the intermediate generated in step (a) with a Lewis acid selected from the group consisting of FeCl₃ and AlCl₃ under suitable conditions to form the alcohol compound having the structure:

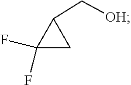

and,
(c) reacting said alcohol compound with an oxidizing agent under suitable conditions to form the carboxylic acid.

E12. The method according to any one of E7 to E11, wherein R₇ is C₅H₁₁, and R₆ is methyl or ethyl.

E13. The method according to any one of E7 to E12, wherein the reaction is carried out using n-Bu₄NBr, n-Bu₄NI or n-Bu₄NOH.

E14. The method according to any one of E7 to E13, wherein the Lewis acid is FeCl₃.

E15. The method according to any one of E7 to E14, wherein the oxidizing agent is selected from periodate, chromate, peroxide, sodium hypochlorite and potassium hypochlorite.

E16. The method according to any one of E7 to E15, wherein the oxidizing agent is sodium hypochlorite.

E17. The method of E7, wherein the carboxylic acid is prepared by (a) treating a compound having the structure:

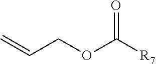

wherein R₇ is C₄-C₆ alkyl C₁-C₆ alkyl, C₃-C₆ cycloalkyl, benzyl, C₆-C₁₂ aryl, or C₄-C₉ heteroaryl with an ester compound having the structure:

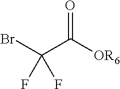

wherein R₆ is selected from the group consisting of C₁-C₅ alkyl, benzyl, C₆-C₁₂ aryl, and C₄-C₉ heteroaryl under suitable conditions to form an intermediate having the structure:

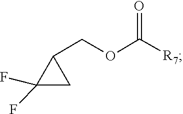

(b) treating the intermediate generated in step (a) with a base selected from the group consisting of sodium hydroxide, potassium hydroxide, lithium hydroxide, calcium hydroxide, cesium carbonate, lithium carbonate, potassium carbonate, ammonium sodium carbonate, ammonium carbonate, lithium bicarbonate, sodium bicarbonate, potassium carbonate, metal or ammonium carboxylates, mono-, and di- and tri-basic metal phosphates, under suitable conditions to form an alcoholic compound having the structure:

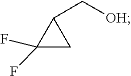

(c) reacting said alcoholic compound with an oxidizing agent selected from the group consisting of periodate, chromate, peroxide, sodium hypochlorite and potassium hypochlorite under suitable conditions to provide the carboxylic acid.

E18. The method according to E17, wherein R₇ is C₅H₁₁, and R₆ is methyl or ethyl.

E19. The method according to any of E17 or E18, wherein the reaction is carried out using n-Bu₄NBr.

E20. The method according to any one of E17 to E19, wherein the base is potassium hydroxide.

E21. The method according to any one of E17 to E20, wherein the oxidizing agent is selected from periodate, chromate, peroxide, sodium hypochlorite and potassium hypochlorite.

E22. The method according to any one of E17 to E21, wherein the oxidizing agent is sodium hypochlorite.

E23. A compound of formula III:

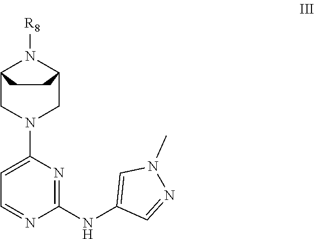

or a salt thereof, or solvate thereof, wherein R₈ is optionally substituted arylmethylene, and said salt is a dihydrochloride or dihydrobromide salt.

E24. The compound of E23 wherein R₈ is benzyl and the salt is a dihydrochloride.

E25. The compound of any one of E23 or E24, or a salt thereof, wherein said solvate is a hydrate.

E26. A method for preparing a compound of formula III:

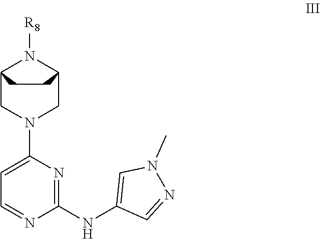

or a salt thereof, or solvate thereof, wherein R₈ is optionally substituted arylmethylene, and said salt is a dihydrochloride or dihydrobromide salt, comprising the step of reacting a compound having the structure:

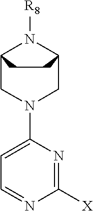

wherein R₈ is optionally substituted arylmethylene, and X is methoxy, ethoxy, Cl, Br or I, with a compound having the structure:

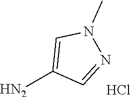

under conditions suitable to form the compound of formula III.

E27. The method of E1, wherein the compound of formula IV:

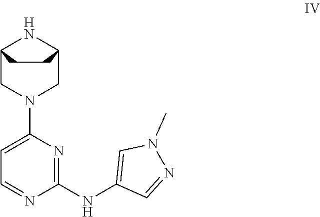 [SMILES: Cn1cc(Nc2nccc(N3C[C@H]4CC[C@@H](C3)N4)n2)cn1]

is prepared under suitable conditions from a compound of formula III:

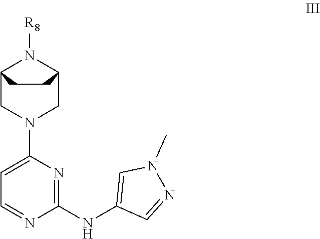

or a salt thereof, or solvate thereof, wherein R₈ is optionally substituted arylmethylene, and said salt is a dihydrochloride or dihydrobromide salt.

E28. The method of E27, wherein R₈ is benzyl and said suitable conditions comprise a hydrogenating or reducing agent.

E29. The method of E4, wherein the compound of formula IV:

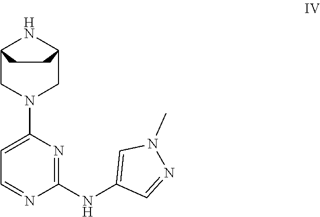 [SMILES: Cn1cc(Nc2nccc(N3C[C@H]4CC[C@@H](C3)N4)n2)cn1]

is prepared under suitable conditions from a compound of formula III:

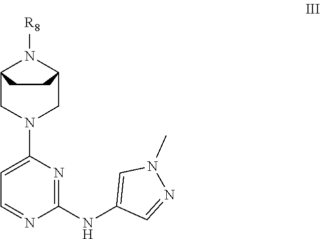

or a salt thereof, or solvate thereof, wherein R₈ is optionally substituted arylmethylene, and said salt is a dihydrochloride or dihydrobromide salt.

E30. The method of E29, wherein R₈ is benzyl and said suitable conditions comprise a hydrogenating or reducing agent.

E31. A compound of formula I:

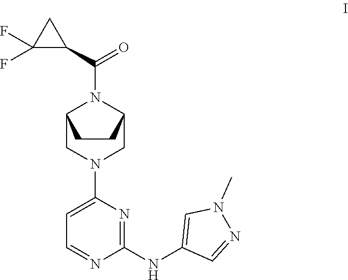 [SMILES: Cn1cc(Nc2nccc(N3C[C@H]4CC[C@@H](C3)N4C(=O)[C@@H]3CC3(F)F)n2)cn1]

prepared by a process comprising (a) (i) preparing a salt from a compound having the structure:

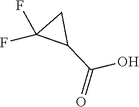

and a base having the structure:

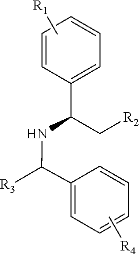

wherein R₁, R₂, R₃, and R₄ are each independently selected from the group consisting of hydrogen, halo, hydroxy, C₁-C₆ alkyl and C₁-C₆ alkoxy; or,
(ii) preparing an activated ester having the structure:

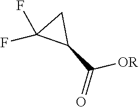

wherein R is selected from the group consisting of C₆-C₁₂ aryl and C₄-C₉ heteroaryl, wherein said C₆-C₁₂ aryl and C₄-C₉ heteroaryl are optionally substituted with a cyano, —S(═O)—R₀, —S(═O)₂—R₀, nitro, C₁-C₆ alkoxy, or halo, where R₀ is C₁-C₆ alkyl;
(b) reacting said salt with a compound of formula IV:

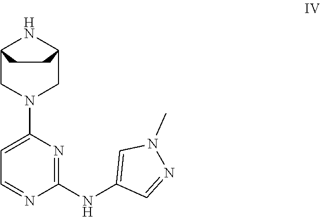 [SMILES: Cn1cc(Nc2nccc(N3C[C@H]4CC[C@@H](C3)N4)n2)cn1]

under conditions suitable to form the compound of formula I.

E32. The compound of E31, or a salt thereof, wherein R₁, R₂, R₃, and R₄ are hydrogen.

E33. The compound according to any one of E31 or E32, or a salt thereof, wherein R isp-cyanophenyl or isoquinolin-3-yl.

E34. The compound according to any one of E31 to E33, wherein the compound of formula IV:

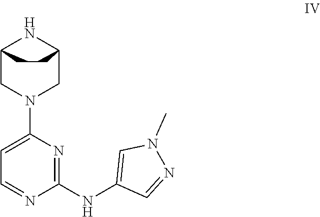 [SMILES: Cn1cc(Nc2nccc(N3C[C@H]4CC[C@@H](C3)N4)n2)cn1]

is prepared under suitable conditions from a compound of formula III:

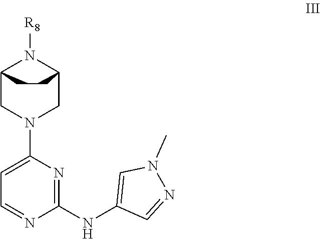

or a salt thereof, wherein R₈ is optionally substituted arylmethylene, and said salt is a dihydrochloride or dihydrobromide salt.

E35. The compound according to any one of E31 to E34, or a salt thereof, wherein R₈ is benzyl and said suitable conditions comprise a hydrogenating or reducing agent.

E36. The p-toluenesulfonic acid salt of a compound of formula I:

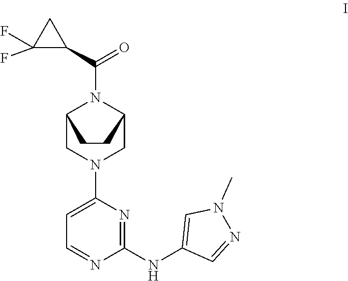 [SMILES: Cn1cc(Nc2nccc(N3C[C@H]4CC[C@@H](C3)N4C(=O)[C@@H]3CC3(F)F)n2)cn1]

prepared by a process comprising (a) (i) preparing a salt from a compound having the structure:

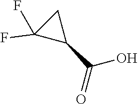

and a base having the structure:

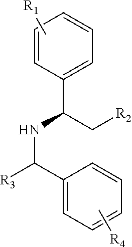

wherein R₁, R₂, R₃, and R₄ are each independently selected from the group consisting of hydrogen, halo, hydroxy, C₁-C₆ alkyl and C₁-C₆ alkoxy; or,
(ii) preparing an activated ester having the structure:

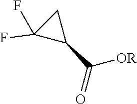

wherein R is selected from the group consisting of C₆-C₁₂ aryl and C₄-C₉ heteroaryl, wherein said C₆-C₁₂ aryl and C₄-C₉ heteroaryl are optionally substituted with a cyano, —S(═O)—R₀, —S(═O)₂—R₀, nitro, C₁-C₆ alkoxy, or halo, where R₀ is C₁-C₆ alkyl;
(b) reacting said salt with a compound of formula IV:

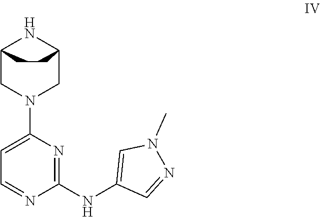 [SMILES: Cn1cc(Nc2nccc(N3C[C@H]4CC[C@@H](C3)N4)n2)cn1]

under suitable conditions to form the compound of formula I:

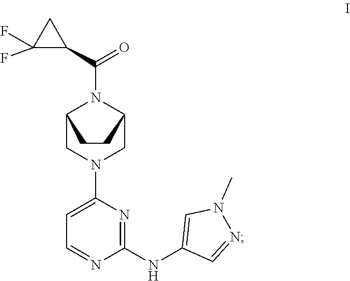 [SMILES: Cn1cc(Nc2nccc(N3C[C@H]4CC[C@@H](C3)N4C(=O)[C@@H]3CC3(F)F)n2)cn1]

and,
(c) treating said compound with p-toluenesulfonic acid under suitable conditions to afford the p-toluenesulfonic acid salt of formula IA:

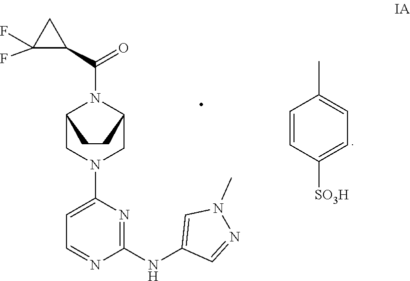 [SMILES: Cn1cc(Nc2nccc(N3C[C@H]4CC[C@@H](C3)N4C(=O)[C@@H]3CC3(F)F)n2)cn1 ; Cc1ccc(S(=O)(=O)O)cc1]

E37. The p-toluenesulfonic acid salt prepared in accordance with E36, wherein R₁, R₂, R₃, and R₄ are hydrogen.

E38. The p-toluenesulfonic acid salt prepared in accordance with any one of E36 or E37, wherein R isp-cyanophenyl or isoquinolin-3-yl.

E37. A method of preparing a compound having the structure:

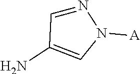

wherein A is C₁-C₆ alkyl, comprising (a) reacting a compound having the structure:

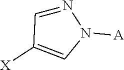

wherein X is halo, with acetamide in the presence of catalyst under suitable conditions to prepare a protected compound having the formula:

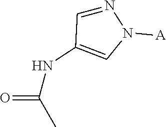

and (b) treating said protected compound under suitable conditions to form the compound having the structure:

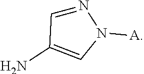

E38. The method of E37, wherein A is methyl and X is bromo.

E39. The method of any one of E37 or E38, wherein the catalyst is CuI and a ligand selected from the group consisting of rac-trans-N,N′-dimethylcyclohexane-1,2-diamine and N,N-dimethylethylenediamine.

E40. The method of claim any one of E37 to E39, wherein step (b) is conducted in acidic conditions.

### Synthetic Methods

The following schemes and written descriptions provide general details regarding the preparation of the compound of formula I, or the p-toluenesulfonic acid salt thereof. In particular, the compound or salt can be prepared by the procedures described by reference to the Schemes that follow, or by the specific methods described in the Examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of the compound of formula I, or the p-toluenesulfonic acid salt thereof.

In addition, the skilled person will appreciate that it may be necessary or desirable at any stage in the synthesis of the compound of formula I, or the p-toluenesulfonic acid salt thereof, to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

Accordingly, the compound of formula I, or the p-toluenesulfonic acid salt thereof can be prepared by the procedures described in the general methods presented below or by routine modifications thereof. The present invention also encompasses any one or more of these processes for preparing the derivatives of formula I, in addition to any novel intermediates used therein. The person skilled in the art will appreciate that the following reactions may be heated thermally or under microwave irradiation. It will be further appreciated that it may be necessary or desirable to carry out the transformations in a different order from that described in the schemes, or to modify one or more of the transformations, to provide the desired compound of the invention.

One skilled in the art will also recognize that some compounds of the invention are chiral and thus may be prepared as racemic or scalemic mixtures of enantiomers. Several methods are available and are well known to those skilled in the art for the separation of enantiomers. A preferred method for the routine separation enantiomers is supercritical fluid chromatography employing a chiral stationary phase.

The compound of formula I or the p-toluenesulfonic acid salt thereof, may be prepared from compounds A-1, A-2 and C-3, as illustrated by Scheme A. Compounds of formulae A-1, A-2 and C-3 are commercially available or may be synthesized by those skilled in the art according to the literature or preparations described herein. For these purposes, PG is a protecting group, as known by those so skilled in the art, for example, and may be tert-butoxycarbonyl. Compounds of formula A-3 may be prepared from compounds of formulae A-1 and A-2 according to process step (i), an aromatic nucleophilic substitution reaction in the presence of an organic base. Preferred conditions comprise triethylamine in methanol at from 0° C. to room temperature. This reaction (i) can be run in various solvents, including methanol, 2-MeTHF, DMSO, THF or combinations thereof. Organic bases used in the reaction include such bases as tertiary amines, DBN, guanidine, amidine, NML, potassium carbonate, potassium phosphate, lithium hydroxide, lithium methoxide, and lithium carbonate.

Compounds of formula A-5 may be prepared from compounds of formula A-3 according to process steps (ii) and (iii), a nucleophilic substitution reaction with compounds of formula C-3 under either Buchwald-Hartwig cross coupling conditions or mediated by acid and elevated temperatures followed by a deprotection reaction mediated by either an inorganic or organic acid. Typical Buchwald-Hartwig conditions comprise a suitable palladium catalyst with a suitable chelating phosphine ligand with an inorganic base in a suitable organic solvent at elevated temperatures either thermally or under microwave irradiation. Preferred conditions comprise either a) palladium(II) acetate and 2-dicyclohexylphosphino-2′,4′,6′-triisopropylbiphenyl or xantphos with sodium tert-butoxide, b) potassium phosphate or cesium carbonate in DMA at from 120-140° C. under microwave irradiation or c) BrettPhos Pd G3 with cesium carbonate as base and either DMA or dioxane as solvent at 40° C. Typical acidic conditions comprise a suitable inorganic acid in a suitable alcoholic solvent at elevated temperatures either thermally or under microwave irradiation. Preferred conditions comprise concentrated hydrochloric acid in iso-propanol at 140° C. under microwave irradiation. Alternatively, the deprotection occurs in situ during process step (ii). Compounds of formula A-6 may be prepared from compounds of formula A-5 according to process step (iv), an amide bond formation reaction with compounds of formula BC(O)X, wherein X may be chloro, hydroxy, a suitable leaving group or anhydride (e.g., (S)-2,2-Difluorocyclopropane-1-carboxylic acid). Wherein compounds of formula BC(O)X are acid chlorides (e.g., Example 2), preferred conditions comprise triethylamine in dichloromethane at room temperature. Wherein compounds of formula BC(O)X are carboxylic acids (e.g., Example 1) activation of the carboxylic acid using a suitable organic base and a suitable coupling agent is employed. Preferred conditions comprise DIPEA or triethylamine with HATU in dichloromethane or DMF at room temperature. Many other amide bond-forming reagents work for this transformation including the acid chloride, CDI/HOPO, T3P, EDCl, and DPPCl. Trifluoroethanol is a good alternative solvent.

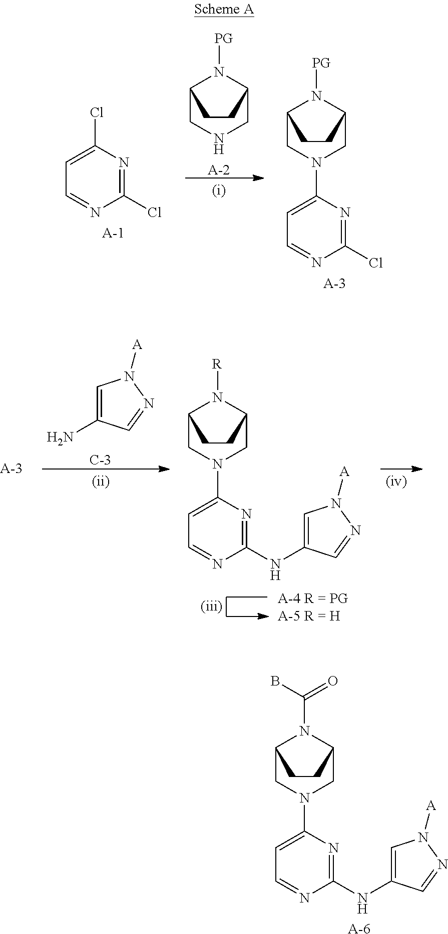

Alternatively, the compound of formula I, or the p-toluenesulfonic acid salt thereof, may be prepared from compounds A-3 and C-3, as illustrated by Scheme B. Compounds of formula A-3 are prepared as described in Scheme A. Compounds of formula C-3 are commercially available or may be synthesized by those skilled in the art according to the literature or preparations described herein. Compounds of formula B-1 may be prepared from compounds of formula A-3 according to process step (i) a deprotection reaction mediated by either an inorganic or organic acid in a suitable organic solvent. Preferred conditions comprise hydrochloric acid or TFA in dioxane or DCM. This reaction can be run in trifluoroethanol. Other acids can also be used such as acetic acid, phosphoric acid, citric, L-tartaric, methane sulfonic acid, and sulfuric acid.

Compounds of formula B-2 may be prepared from compounds of formulae B-1 and BC(O)X according to process step (ii), an amide bond formation reaction as described in Scheme A. Compounds of formula A-6 may be prepared from compounds of formula B-2 according to process step (iii), a nucleophilic substitution reaction with compounds of formula C-3 under either Buchwald-Hartwig cross coupling conditions or mediated by acid and high temperatures as described in Scheme A.

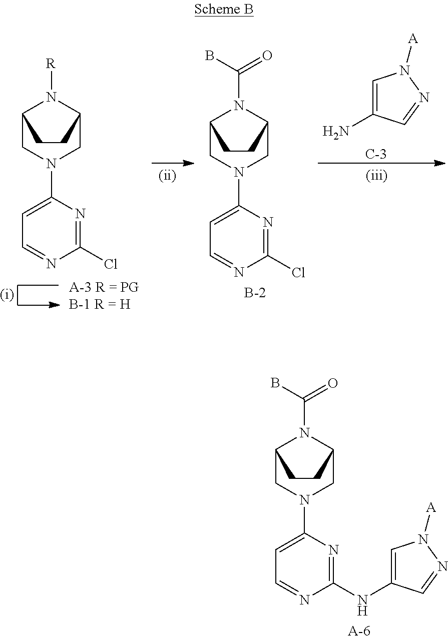

Compounds of formula C-3 employed in Scheme A and Scheme B may be prepared from compounds of formula C-1, as illustrated in Scheme C. The compound of formula C-1 is commercially available or may be synthesized by those skilled in the art according to the literature or preparations described herein. Compounds of formula C-2 may be prepared from compounds of formula C-1 according to process step (i) an alkylation reaction with an appropriately substituted alkyl halide of the formula AX where X is Cl, Br or I in the presence of an inorganic or organic base and a solvent such as DMF, or an addition reaction to an epoxide in the presence of an inorganic or organic base. Compounds of formula C-3 may be prepared from compounds of formula C-2 according to process step (ii) a reduction typically performed in the presence of a metal catalyst such as palladium or nickel, hydrogen gas at a pressure of 1-50 atmospheres, and a protic solvent such as methanol.

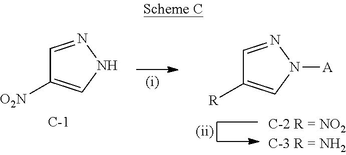

### PREPARATIONS AND EXAMPLES

The following non-limiting Preparations and Examples illustrate the preparation of compounds and salts of the present invention. In the Examples and Preparations that are set out below, and in the aforementioned Schemes, the following abbreviations, definitions and analytical procedures may be referred to. Other abbreviations common in the art may also be used. Compounds of the present invention were named using ChemDraw Professional™ version 18.0 (Perkin Elmer) or were given names which appeared to be consistent with IUPAC nomenclature.

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g., s, singlet; d, doublet; t, triplet; q, quartet; m, multiplet; br, broad. The following abbreviations have been used for common NMR solvents: CD₃CN, deuteroacetonitrile; CDCl₃, deuterochloroform; DMSO-d₆, deuterodimethylsulfoxide; and CD₃OD, deuteromethanol. Where appropriate, tautomers may be recorded within the NMR data; and some exchangeable protons may not be visible. Some resonances in the NMR spectrum appear as complex multiplets because the isolate is a mixture of two conformers.

Mass spectra were recorded using electron impact ionization (EI), electrospray ionization (ESI) or atmospheric pressure chemical ionization (APCI). The observed ions are reported as MS m/z and may be positive ions of the compound [M]⁺, compound plus a proton [MH]⁺, or compound plus a sodium ion [MNa]⁺. In some cases the only observed ions may be fragment ions reported as [MH-(fragment lost)]⁺. Where relevant, the reported ions are assigned for isotopes of chlorine (³⁵Cl and/or ³⁷Cl), bromine (⁷⁹Br and/or ⁸¹Br) and tin (¹²⁰Sn). Wherein TLC, chromatography or HPLC has been used to purify compounds, one skilled in the art may choose any appropriate solvent or combination of solvents to purify the desired compound. Chromatographic separations (excluding HPLC) were carried out using silica gel adsorbent unless otherwise noted.

All reactions were carried out using continuous stirring under an atmosphere of nitrogen or argon gas unless otherwise noted. In some cases, reactions were purged with nitrogen or argon gas prior to the start of the reaction. In these cases, the nitrogen or argon gas was bubbled through the liquid phase of the mixture for the approximate specified time. Solvents used were commercial anhydrous grades. All starting materials were commercially available products. In some cases, the Chemical Abstracts Service® (CAS) identification number is provided to assist with clarity. It will be apparent to one skilled in the art that the word “concentrated” as used herein generally refers to the practice of evaporation of solvent under reduced pressure, typically accomplished using a rotary evaporator.

The following abbreviations are used herein:
ACN: acetonitrile;
BrettPhos Pd G3: [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate;
CDI: 1,1′-carbonyldiimidazole;
Cs₂CO₃: cesium carbonate;
DMF: N, N-dimethylformamide;
ESI: electrospray ionization;
EtOAc: ethyl acetate;
g: gram;
HPLC: high pressure liquid chromatography;
HRMS: high resolution mass spectrum;
KOH: potassium hydroxide;
MeOH: methanol;
MIBK: methyl isobutyl ketone;
mg: milligram;
mL: milliliter;
mmol: millimole;
Mpa: megapascal;
MTBE: methyl tert-butyl ether;
Pd/C: palladium on carbon;
THF: tetrahydrofuran;
T3P: 2,4,6-tripropyl-1,3,5,2,4,6-trioxatriphosphorinane-2,4,6-trioxide.

### Preparation 1: 4-((1R,5S)-8-Benzyl-3,8-diazabicyclo[3.2.1]octan-3-yl)-N-(1-methyl-1H-pyrazol-4-yl)pyrimidin-2-amine

### (a) (1R,5S)-8-Benzyl-3-(2-chloropyrimidin-4-yl)-3,8-diazabicyclo[3.2.1]octane

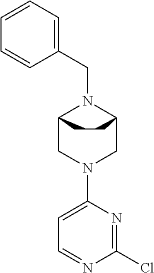 [SMILES: Clc1nccc(N2C[C@H]3CC[C@@H](C2)N3Cc2ccccc2)n1]

(1R,5S)-8-Benzyl-3,8-diazabicyclo[3.2.1]octane (5.0 g, 18 mmol) is dissolved in methanol (50 mL) and 2-methyl tetrahydrofuran (25 mL) and the solution is cooled to 0° C. 2,4-Dichloropyrimidine (2.99 g, 20 mmol) is added. N,N-diisopropylethylamine (10.8 mL, 62 mmol) is added. The reaction is stirred until complete. The solution is warmed to room temperature. Water (50 mL) is added and the reaction heated to 55° C. Reaction may be seeded. Reaction is cooled and the product is isolated by filtration and dried under vacuum. (1R,5S)-8-Benzyl-3-(2-chloropyrimidin-4-yl)-3,8-diazabicyclo[3.2.1]octane (5.3 g) is isolated as a crystalline white solid. ¹H NMR (400 MHz, DMSO) δ 8.06 (d, J=6.1 Hz, 1H), 7.59-6.97 (m, 5H), 6.72 (d, J=6.2 Hz, 1H), 4.15 (s, 1H), 3.56 (s, 3H), 3.26 (s, 2H), 3.07 (s, 2H), 1.99 (dd, J=8.8, 4.2 Hz, 2H), 1.49 (t, J=7.2 Hz, 2H). ¹³C NMR (101 MHz, DMSO) δ 164.2, 159.8, 157.5, 139.8, 128.9, 128.6, 127.3, 102.7, 57.9, 56.0, 25.6. mp: 117.6° C. Alternate Procedure: (1R,5S)-8-Benzyl-3,8-diazabicyclo[3.2.1]dihydrochloride (5 g, 18.17 mmol) and methanol (25 mL) are charged to a 50 mL reactor. N,N-diisopropylethylamine (9.8 mL, 56 mmol, 3.1 equiv) was added to the slurry. A solution of 2,4-dichloropyrimidine (2.6 g, 17 mmol, 0.96 equiv) in 2-methyltetrahydrofuran (25 mL) and methanol (5 mL) was added over 30 min. The reaction is stirred until complete and carried directly into the Step 2 procedure.

### (b) 4-((1R,5S)-8-Benzyl-3,8-diazabicyclo[3.2.1]octan-3-yl)-N-(1-methyl-1H-pyrazol-4-yl)pyrimidin-2-amine

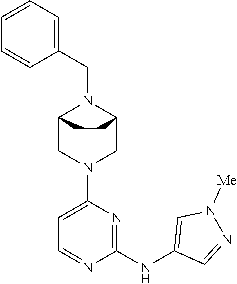 [SMILES: Cn1cc(Nc2nccc(N3C[C@H]4CC[C@@H](C3)N4Cc3ccccc3)n2)cn1]

(1R,5S)-8-Benzyl-3-(2-chloropyrimidin-4-yl)-3,8-diazabicyclo[3.2.1]octane (24 g, 76.2 mmol) is dissolved in methanol (175 ml) and 2-methyl tetrahydrofuran (110 mL) and water (12 mL) is added. 1-Methyl-1H-pyrazol-4-amine hydrochloride (12.2 g, 91.5 mmol, 1.2 equiv) is charged. The solution is heated to 60° C. until reaction is complete. The reaction is cooled to 45° C. and water (190 mL) is added. Aqueous potassium hydroxide 45 wt % (16 mL) is added. The reaction may be seeded. The slurry is cooled to 15° C. The product is isolated by filtration. 4-((1R,5S)-8-Benzyl-3,8-diazabicyclo[3.2.1]octan-3-yl)-N-(1-methyl-1H-pyrazol-4-yl)pyrimidin-2-amine (85% yield) is isolated as a white crystalline solid. ¹H NMR (400 MHz, DMSO) δ 8.80 (s, 1H), 7.88 (d, J=5.9 Hz, 1H), 7.73 (s, 1H), 7.42 (t, J=4.0 Hz, 3H), 7.38-7.30 (m, 2H), 7.26 (t, J=7.3 Hz, 1H), 6.03 (d, J=6.0 Hz, 1H), 3.88 (s, 2H), 3.76 (d, J=1.3 Hz, 3H), 3.57 (s, 2H), 3.33 (s, 3H), 3.03 (d, J=11.9 Hz, 2H), 1.99 (dd, J=7.6, 3.7 Hz, 2H), 1.55 (t, J=6.9 Hz, 2H). ¹³C NMR (101 MHz, DMSO) δ 163.9, 159.4, 156.7, 139.9, 130.0, 128.9, 128.6, 127.2, 124.3, 120.2, 94.1, 58.2, 56.2, 50.5, 39.0, 25.7. mp: 153.6° C. Alternate Procedure: To the solution from step 1 is added methanol (15 mL) and water (2.5 mL). 1-Methyl-1H-pyrazol-4-amine hydrochloride (2.8 g, 1.2 equiv, 21 mmol) is added and the reaction heated to 65° C. until reaction completion. The reaction is cooled to 45° C. A 1M aqueous solution of potassium hydroxide (46 mL, 46 mmol) is added. The solution may be seeded. The slurry is cooled to 15° C. The product is isolated by filtration. The solid was dried in a vacuum oven, providing 4-((R,5S)-8-benzyl-3,8-diazabicyclo[3.2.1]octan-3-yl)-N-(1-methyl-1H-pyrazol-4-yl)pyrimidin-2-amine (5.2 g, 14 mmol, 82% yield).

### Preparation 2. 4-((1R,5S)-3,8-Diazabicyclo[3.2.1]octan-3-yl)-N-(1-methyl-1H-pyrazol-4-yl)pyrimidin-2-amine

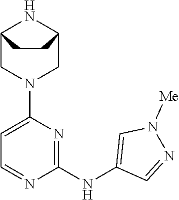 [SMILES: Cn1cc(Nc2nccc(N3C[C@H]4CC[C@@H](C3)N4)n2)cn1]

4-((1R,5S)-8-Benzyl-3,8-diazabicyclo[3.2.1]octan-3-yl)-N-(1-methyl-1H-pyrazol-4-yl)pyrimidin-2-amine (10 g, 23.9 mmol) is combined with water (25 mL) and concentrated hydrochloric acid (3.53 mL, 43.02 mmol, 1.8 equiv). Isopropanol (11 mL) and the pH is adjusted to pH 4 using 1 M aq. HCl solution (4.78 mL, 4.78 mmol, 0.2 equiv). Palladium hydroxide (10%) on carbon (0.3 g) is added and the mixture is heated to 40° C. Hydrogen gas is added under pressure and the mixture stirred until reaction completion. The mixture is cooled to 25 and the catalyst filtered. Water (47 mL) and isopropanol (10 mL) are added. A solution of potassium hydroxide in water (2.5 M, 21 mL, 2.2 equiv) is added. The mixture is then isolated by filtration and washed with water. 4-((1R,5S)-3,8-Diazabicyclo[3.2.1]octan-3-yl)-N-(1-methyl-1H-pyrazol-4-yl)pyrimidin-2-amine is isolated as a crystalline white solid. ¹H NMR (400 MHz, DMSO) δ 8.77 (s, 1H), 7.86 (d, J=5.9 Hz, 1H), 7.72 (s, 1H), 7.44 (s, 1H), 6.01 (d, J=6.0 Hz, 1H), 3.85 (s, 2H), 3.77 (s, 3H), 3.52-3.46 (m, 2H), 2.93 (d, J=11.9 Hz, 2H), 1.66 (dd, J=8.2, 4.3 Hz, 2H), 1.54 (t, J=6.5 Hz, 2H). ¹³C NMR (101 MHz, DMSO) δ 164.0, 159.3, 156.6, 130.0, 124.3, 120.3, 94.1, 53.5, 51.3, 39.0, 28.9. mp: 243.2° C.

### Preparation 3. (S)-2,2-difluorocyclopropane-1-carboxylic acid 2,2′,2″-nitrilotris(ethan-1-ol) salt

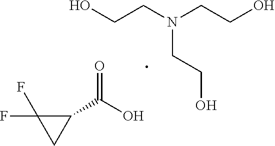

To a 100 mL reactor was added ACN (50.0 mL) and triethanolamine (12.2 g, 1.0 equivs). This solution was heated to 45° C., and a pre-mixed solution of (S)-2,2-difluorocyclopropane-1-carboxylic acid prepared as describe in preparation 68 of U.S. Pat. No. 9,663,526 (10.1 g, 1.0 equiv) in MTBE (50.0 mL, ~20% w/w) was added dropwise over 100 minutes. After addition, the reaction was held at 45° C. for 30 minutes, then cooled to 20° C. at a rate of 0.25° C./min. The mixture was granulated for 30 minutes, then filtered and washed with MTBE (40.0 mL), and dried under vacuum at 50° C. ¹H NMR (400 MHz, DMSO-d₆) δ 6.85 (s, 4H), 3.61 (t, J=5.7 Hz, 6H), 2.97 (t, J=5.7 Hz, 6H), 2.38 (ddd, J=15.4, 10.8, 7.9 Hz, 1H), 1.84-1.62 (m, 2H). ¹³C NMR (101 MHz, DMSO-d₆) δ 169.0, 115.9, 113.1 (dd, J=285.9, 281.2 Hz), 113.1, 110.3, 57.4, 56.5, 27.7, 27.6 (dd, J=12.0, 9.2 Hz), 27.6, 27.5, 16.2, 16.1 (t, J=9.8 Hz), 16.0. mp: 82.4° C.

### Preparation 4. 2,2-Difluorocyclopropane-1-(S)-carboxylate (R)—N-Benzyl-1-phenylethan-1-aminium Salt

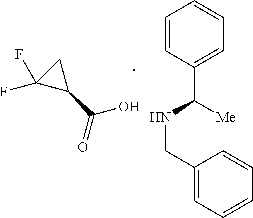

To a 250 mL vessel was added MTBE (134 mL), (S)-2,2-difluorocyclopropane-1-carboxylic acid 2,2′,2″-nitrilotris(ethan-1-ol) salt (20.0 g, 1.0 equiv), and a premixed solution of sulfuric acid (4.3 mL, 1.1 equivs) in water (86.0 mL). The mixture was stirred until all solids dissolved, and then the layers allowed to settle. The layers were separated, and the bottom (aqueous) layer was back-extracted with MTBE (58 mL). The combined organic layers were dried via azeotropic distillation to achieve a final concentration of (S)-2,2-difluorocyclopropane-1-carboxylic acid of ~15% (w/w) in MTBE. To this solution was added chiral amine (R)-(+)—N-benzyl-a-methylbenzylamine (13.0 g, 0.85 equivs) dropwise over ~1 hour. After ~25% of the amine had been added, the reaction was seeded with previously purified (R)—N-benzyl-1-phenylethan-1-amine (S)-2,2-difluorocyclopropane-1-carboxylate (50 mg, 0.002 equivs). After addition of the amine, the slurry was allowed to granulate, then filtered, and washed with MTBE (12.0 mL) that had been pre-chilled to 10° C., and the solids dried under vacuum at 50° C. The crude solids (10.57 g) were returned to the same vessel and MeCN (35.0 mL) added. The slurry was heated to 80° C. to fully dissolve the solids. The solution was cooled to 22° C. at a rate of 0.2° C./min and allowed to granulate. The product was collected by filtration and washed with MeCN (13.0 mL) before drying under vacuum at 50° C. ¹H NMR (400 MHz, DMSO-d₆) δ 9.25 (s, 2H), 7.46 (d, J=6.9 Hz, 2H), 7.43-7.24 (m, 9H), 4.00 (q, J=6.7 Hz, 1H), 3.74 (d, J=13.3 Hz, 1H), 3.65 (s, 1H), 2.50-2.39 (m, 1H), 1.90-1.66 (m, 2H), 1.43 (d, J=6.7 Hz, 3H). ¹³C NMR (101 MHz, DMSO-d₆) δ 168.5, 142.4, 137.1, 129.3, 129.0, 128.7, 128.0, 127.9, 127.6, 116.0, 113.2, 113.1 (dd, J=286.1, 281.3 Hz), 110.3, 57.2, 49.8, 27.6, 27.5, 27.5 (dd, J=12.0, 9.3 Hz), 27.4, 22.5, 16.2, 16.1 (t, J=9.8 Hz), 16.07. mp: 138.6° C.

### Preparation 5

### Alternate Procedure: (R)—N-Benzyl-1-phenylethan-1-amine (S)-2,2-difluorocyclopropane-1-carboxylate

To a pre-heated solution of anisole (2.0 kg) at 140° C. was added gradually a solution containing butyl acrylate (200 g), ethyl bromdifluoroacetate (1426 g), and tetrabutylammonium bromide (9.8 g). The mixture was stirred and cooled the mixture was cooled, and the desired product purified by distillation to give butyl 2,2-difluorocyclopropane-1-carboxylate as a solution in anisole. This solution was added to a solution of aqueous NaOH and the biphasic mixture heated to 40° C. The mixture was cooled and filtered through Celite™ and layers were separated. The aqueous layer was washed with MTBE, acidified to pH 1-2, and washed twice with MTBE. The combined organic phases were distilled to solvent swap to MeCN to give 2,2-difluorocyclopropane-1-carboxylic acid as a solution in MeCN. To this solution was added chiral amine (R)—N-benzyl-1-phenylethan-1-amine and the mixture was heated to 40°, MTBE was added, and the mixture was cooled to 10° C. leading to crystallization of the desired 2,2-difluorocyclopropane-1-(S)-carboxylate (R)—N-benzyl-1-phenylethan-1-aminium salt. The solids were isolated by filtration and recrystallized from MeCN to give the material containing NMT 0.5% of the desired acid enantiomer.

### Preparation 6

### Alternate Procedure: (R)—N-Benzyl-1-phenylethan-1-amine (S)-2,2-difluorocycloprop-ane-1-carboxylate

To a preheated eutectic mixture of 26.5% m/m biphenyl in diphenyl ether (1497 g) at 130° C. was added a solution of tetrabutylammonium bromide (6.8 g), butyl acrylate (1615 g), ethyl bromodifluoroacetate (853 g), in a eutectic mixture of biphenyl in diphenyl ether (748 g) and the mixture was maintained at 130-135° C. Additional tetrabutylammonium bromide (6.8 g) is added. The reaction is cooled, and 1,2-dichlorobenzene (1976 g) is added, and the mixture was distilled to provide the desired butyl 2,2-difluorocyclopropane-1-carboxylate as a solution in 1,2-dichlorobenzene. To this solution is added tetrabutylammonium bromide (5 mol %) and aqueous NaOH (4 equiv of 15% m/m) and the biphasic mixture stirred at room temperature. Additional tetrabutylammonium bromide is added (1.25 mol %). The phases are separated, and the aqueous phase is washed with MTBE and acidified to pH 1 with aqueous HCl, and extracted twice with MTBE. The combined organic phases are solvent swapped to MeCN to yield a 15% m/m solution of 2,2-difluorocyclopropane-1-carboxylic acid. The solution is heated to 40° C. and (R)-(+)—N-benzyl-1-phenylethan-1-amine (1.1 equiv) is added. The reaction is then further heated to 80° C., and the reaction mixture is seeded to promote crystallization of the title salt and cooled. The solids are isolated by filtration, washed with MeCN, and recrystallized using MeCN to give the desired material containing NMT 0.5% of the undesired acid enantiomer.

### Preparation 7

### Alternate Procedure: (R)—N-Benzyl-1-phenylethan-1-amine (S)-2,2-difluorocycloprop-ane-1-carboxylate

To a solution of allyl hexanoate (225 L, 1.28 mol) in anisole (400 L) at 130° C. is added a mixture of ethyl bromodifluoroacetate (328 L, 2.56 mol) and tetrabutylammonium bromide (2.06 L, 0.006 mol) gradually. Additional portion of EBDFA (82.1 L, 0.64 mol) and tetrabutylammonium bromide (2.00 L, 0.006 mol) are added until full conversion of the allyl hexanoate is observed. The mixture is cooled, THF (1600 L) added, and the THF distilled from the system to remove any volatile by-products and give (2,2-difluorocyclopropyl)methyl hexanoate as a solution in anisole. To this solution is added aqueous KOH (301 L of 48 wt % KOH in 470 L of H₂O) and the mixture heated to 60° C. 0.5 wt % K₂HPO₄ (100 L) is added and the layers are separated. The aqueous layer is washed twice with methylene chloride (600 mL, each). To the combined organic layers is added TEMPO (9.94 g, 0.064 mol), potassium bromide (75.7 g, 0.636 mol), sodium bicarbonate (506.7 g, 6.03 mol), tetrabutylammonium bromide (20.51 L, 0.064 mol), and water (397.6 L) to forma a biphasic mixture. The reaction mixture is cooled to 10° C. and 14.8 wt % NaOCl (1777.5 L, 4.07 mol) is added. Sodium thiosulfate (100.6 g, 0.636 mol) is added, the solids are removed by filtration, and the cake is rinsed with methylene chloride (179 L). The layers are separated, and the organic layer is washed with aqueous NaOH (4.51 L of 30% NaOH in 795 L of H₂O). The pH of the aqueous layer is adjusted with HCl to pH 2 and extracted twice with methylene chloride (596 L, each).

The aqueous layer is washed with methyl tert-butyl ether twice (600 L, each). The solvent is swapped to give a solution of 2,2-difluorocyclopropane-1-carboxylic acid in acetonitrile. To this solution is added chiral amine (R)—N-benzyl-1-phenylethan-1-amine, and the reaction is stirred at 40° C. and then cooled to 20° C. which leads to crystallization of the title salt. The solids are collected by filtration, washed twice with MeCN, and then recrystallized in MeCN to give the title salt containing NMT 0.5% of the undesired acid enantiomer.

### Example 1

### ((S)-2,2-Difluorocyclopropyl)-((1R,5S)-3-(2-((1-methyl-1H-pyrazol-4-yl)amino)pyrimidin-4-yl)-3,8-diazabicyclo[3.2.1]octan-8-yl)methanone p-Tosylate

(R)—N-Benzyl-1-phenylethan-1-amine (S)-2,2-difluorocyclopropane-1-carboxylate (65.7 g, 197 mmol) is slurried in methyl t-butyl ether (441 mL) at 25° C. and treated with aqueous sulfuric acid (22 g, 217 mmol, 1.375 equiv). The phases are separated, the aqueous phase washed with methyl t-butyl ether (189 mL) and the organic phases combined. The combined organic phases are concentrated, tetrahydrofuran (THF) (550 mL) is added and the solution concentrated. A second addition of tetrahydrofuran (550 mL) and concentration is performed.

The solution of (S)-2,2-difluorocyclopropane-1-carboxylate in THF (approximately 430 mL) is cooled to 0° C. and 1,1′-carbonyldiimidazole (CDI) (36.9 g, 205 mmol, 1.3 equiv). Water (22.5 g) is then added. 2-Hydroxypyridine n-oxide (HOPO) (0.9 g, 7.9 mmol, 0.05 equiv) and 4-((1R,5S)-3,8-diazabicyclo[3.2.1]octan-3-yl)-N-(1-methyl-1H-pyrazol-4-yl)pyrimidin-2-amine (45 g, 158 mmol) are added at 0° C. until reaction is complete. The mixture is then warmed to 55° C. To the reaction is added a solution of p-toluenesulfonic acid monohydrate (52.8 g, 278 mmol, 1.75 equiv) in THF (99 mL). The solution is seeded with the title compound salt. A second portion of p-toluenesulfonic acid monohydrate (79.2 g, 416 mmol, 2.25 equiv) in THF (148 mL) is added over 4 hours. The slurry is then cooled to 10° C. The solid title compound salt is recovered by filtration, washed twice with a pre-mixed solution of 95:5 v/v THF/water (5 volumes), then dried at 50+/−5° C. under vacuum. The title compound salt is isolated as a white crystalline solid (79.8 g, 90%).

### Example 2

### Alternate Procedure: ((S)-2,2-Difluorocyclopropyl)-((1R,5S)-3-(2-((1-methyl-1H-pyrazol-4-yl)amino)pyrimidin-4-yl)-3,8-diazabicyclo[3.2.1]octan-8-yl)methanone p-Tosylate

Chiral 2,2-difluorocyclopropane-1-(S)-carboxylic acid was prepared by salt break of 2,2-difluorocyclopropane-1-(S)-carboxylate (R)—N-Benzyl-1-phenylethan-1-aminium salt. The acid chloride was prepared and reacted with isoquinolin-3-ol to afford isoquinolin-3-yl (S)-2,2-difluorocyclopropane-1-carboxylate. ¹H NMR (400 MHz, DMSO) δ 9.23 (s, 1H), 8.20 (d, J=8.3 Hz, 1H), 8.02 (d, J=8.3 Hz, 1H), 7.83 (dd, J=8.4, 6.8 Hz, 1H), 7.75-7.66 (m, 2H), 3.22 (ddd, J=12.3, 10.9, 7.9 Hz, 1H), 2.35-2.13 (m, 2H). ³C NMR (101 MHz, DMSO) δ 165.8, 153.5, 152.4, 138.3, 131.8, 128.2, 127.9 (d, J=2.6 Hz), 127.8, 126.9, 115.07, 112.2, 112.2 (dd, J=286.3, 284.4 Hz), 111.2, 109.3, 31.7, 28.8, 25.5, 25.4 (dd, J=12.8, 10.1 Hz), 25.3, 25.2, 17.4, 17.3 (t, J=10.0 Hz), 17.2.

4-((1R,5S)-3,8-Diazabicyclo[3.2.1]octan-3-yl)-N-(1-methyl-1H-pyrazol-4-yl)pyrimidin-2-amine (1.50 g, 5.26 mmol) is combined with tetrahydrofuran (21.4 mL) and water (1.13 mL). To the reaction is added isoquinolin-3-yl (S)-2,2-difluorocyclopropane-1-carboxylate (1.57 g, 6.30 mmol) and 2-hydroxypyridine N-oxide (0.029 g, 0.26 mmol). The reaction is stirred at room temperature until reaction is complete. The solution is heated to 50° C. p-Toluenesulfonic acid monohydrate (2.44 g, 12.6 mmol) was dissolved in tetrahydrofuran (7.13 mL) and water (0.375 mL). Approximately 1.7 mL of this solution is added to the reaction. The reaction may be seeded. The remaining acid solution is added. The slurry is cooled to room temperature. The solids are isolated by filtration, washing with THF/water. The title compound salt is isolated as a white crystalline solid (2.72 g). ¹H NMR (400 MHz, DMSO) δ 9.23 (s, 1H), 8.20 (d, J=8.3 Hz, 1H), 8.02 (d, J=8.3 Hz, 1H), 7.83 (dd, J=8.4, 6.8 Hz, 1H), 7.75-7.66 (m, 2H), 3.22 (ddd, J=12.3, 10.9, 7.9 Hz, 1H), 2.35-2.13 (m, 2H). ¹³C NMR (101 MHz, DMSO) δ 165.8, 153.5, 152.4, 138.3, 131.8, 128.2, 127.9 (d, J=2.6 Hz), 127.8, 126.9, 115.07, 112.2, 112.2 (dd, J=286.3, 284.4 Hz), 111.2, 109.3, 31.7, 28.8, 25.5, 25.4 (dd, J=12.8, 10.1 Hz), 25.3, 25.2, 17.4, 17.3 (t, J=10.0 Hz), 17.2. mp: 99.3° C.

### Example 3

### Alternate Procedure: ((S)-2,2-Difluorocyclopropyl)-((1R,5S)-3-(2-((1-methyl-1H-pyrazol-4-yl)amino)pyrimidin-4-yl)-3,8-diazabicyclo[3.2.1]octan-8-yl)methanone p-Tosylate

A reactor is charged with title compound salt (20.0 g, 35 mmol) and methyl isobutylketone (MIBK) (160 mL). A solution of sodium carbonate (4.52 g, 42 mmol, 1.2 equiv) in water (55 mL) is added. The reaction is stirred until a homogenous biphasic mixture is obtained. The aqueous layer is separated and back extracted with MIBK (100 mL). The organic phases are combined and washed with water (55 mL). The organic phase is concentrated to a volume of 100 mL. The solution is heated to 85° C. and heptane (67 mL) is added. The solution may be seeded. The solution is cooled to 25° C. The solids are isolated by filtration and washed with 30% heptane in MIBK. The title compound salt is isolated as a white crystalline solid.

### Example 4

### 1-Methyl-1H-pyrazol-4-amine Hydrochloride

### (a) N-(1-Methyl-1H-pyrazol-4-yl)acetamide

To a 100 mL vessel containing a reflux condenser, temperature probe, and overhead stirrer was added 4-bromo-1-methyl-1H-pyrazole (7.0 g), acetamide (7.47 g, 3 equivs), K₂CO₃ (8.83 g, 1.5 equivs), ligand, and 2-methyl-2-butanol (70.0 mL) to give a yellowish slurry. Mixture was sparged with nitrogen for ~20 minutes to remove oxygen. The vessel was then evacuated and backfilled with nitrogen a total of three times. With a strong sweep of nitrogen, the CuI was added, and the vessel heated to an internal temperature of 100° C. After 26 hours, the reaction cooled to 25° C. The reaction was diluted with a pre-mixed solution of sodium citrate (31.0 g, 2.5 equivs) in water (70 mL), which caused all solids to dissolve. The layers were allowed to settle and the bottom (aqueous) layer removed. The resulting organic layer was extracted again with a pre-mixed solution containing sodium citrate (49.6 g, 4 equivs) in water (70 mL) and the layers separated. The resulting organic layer (~90 mL) was concentrated distilled down under vacuum to ~40 mL, during which solid were observed to crystallize in the vessel, and then toluene (75 mL) added. This organic solution was further concentrated down (from ~115 mL to ~60 mL) and then the mixture cooled to 20° C., filtered and dried under vacuum at 50° C. N-(1-Methyl-1H-pyrazol-4-yl)acetamide was isolated as a light gray to tan solid. ¹H NMR (400 MHz, DMSO) δ 9.89 (s, 1H), 7.84 (s, 1H), 7.36 (s, 1H), 3.77 (s, 3H), 1.97 (s, 3H). ¹³C NMR (101 MHz, DMSO) δ 166.9, 129.8, 122.2, 121.5, 39.0, 23.2. mp: 149.0° C.

### (b) N-(1-Methyl-1H-pyrazol-4-amine Hydrochloride

To a 50 mL vessel topped with a reflux condenser, temperature probe, and overhead stirrer was added N-(1-methyl-1H-pyrazol-4-yl)acetamide (3.97 g), followed by 1-BuOH (32.0 mL), water (2.0 mL), and 12M HCl (2.60 mL, 1.2 equivs). A slight exotherm was observed during addition of HCl. The mixture is heated to an internal temperature of 80° C. and held until full conversion of the starting material is observed (typically within 16-24 hours). The reaction mixture is concentrated via distillation to remove water, during which the desired product PF-05602633-01 is observed to crystallize as shimmery, white flakes. The slurry in cooled down to 20° C., the product isolated via filtration, washed with 1-BuOH and dried under vacuum at 50° C.

### Example 5

### (S)-2,2-Difluorocyclopropane-1-carboxylic Acid 2,2′,2″-nitrilotris(ethan-1-ol) Salt

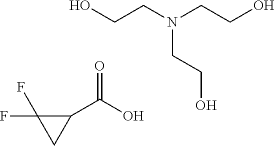

To a 100 mL reactor was added MeCN (50.0 mL) and triethanolamine (12.2 g, 1.0 equivs). This solution was heated to 45° C., and a pre-mixed solution of 2,2-difluorocyclopropane-1-carboxylic (10.1 g, 1.0 equiv) in MTBE (50.0 mL, ~20% w/w) was added dropwise over 100 minutes. After ~40% of the MTBE solution had been added, the reaction was seeded with (S)-2,2-difluorocyclopropane-1-carboxylic acid 2,2′,2″-nitrilotris(ethan-1-ol) salt (42 mg, 0.002 equivs). After addition, the reaction was held at 45° C. for 30 minutes, then cooled to 20° C. at a rate of 0.25° C./min. The mixture was granulated for 30 minutes, then filtered and washed with MTBE (40.0 mL), and dried under vacuum at 50° C. The difluoro acid can then be resolved through crystallization of the diastereomers. ¹H NMR (400 MHz, DMSO) δ 6.85 (s, 4H), 3.61 (t, J=5.7 Hz, 6H), 2.97 (t, J=5.7 Hz, 6H), 2.38 (ddd, J=15.4, 10.8, 7.9 Hz, 1H), 1.84-1.62 (m, 2H). ¹³C NMR (101 MHz, DMSO) δ 169.0, 115.9, 113.1 (dd, J=285.9, 281.2 Hz), 113.1, 110.3, 57.4, 56.5, 27.7, 27.6 (dd, J=12.0, 9.2 Hz), 27.6, 27.5, 16.2, 16.1 (t, J=9.8 Hz), 16.0. mp: 82.4° C.

We claim:

1. A method for preparing a compound of formula I: 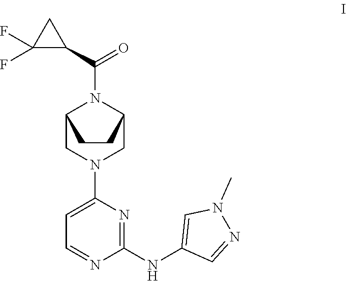 [SMILES: Cn1cc(Nc2nccc(N3C[C@H]4CC[C@@H](C3)N4C(=O)[C@@H]3CC3(F)F)n2)cn1]
comprising
(a) (i) preparing a salt of formula (II) having the structure: 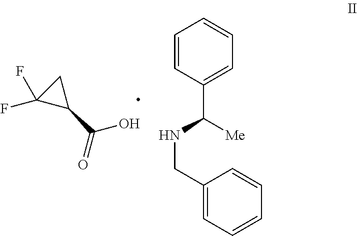 [SMILES: O=C(O)[C@@H]1CC1(F)F ; C[C@@H](NCc1ccccc1)c1ccccc1]
from (A) a compound having the structure: 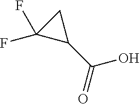
and (B) a base having the structure: 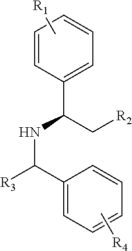
wherein R₁, R₂, R₃, and R₄ are each independently selected from the group consisting of hydrogen, halo, hydroxy, C₁-C₆ alkyl and C₁-C₆ alkoxy,
and isolating from said salt of formula II a chiral compound having the structure: 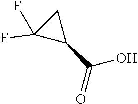
or,
(ii) preparing an activated ester having the structure: 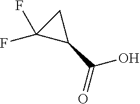
wherein R is selected from the group consisting of C₆-C₁₂ aryl and C₄-C₉ heteroaryl, wherein said C₆-C₁₂ aryl and C₄-C₉ heteroaryl are optionally substituted with a C₁-C₆ alkyl, —S(═O)—R₀, —S(═O)₂—R₀, cyano, nitro, C₁-C₆ alkoxy, or halo, where R₀ is C₁-C₆ alkyl;
(b) reacting said activated ester or said chiral compound with a compound of formula IV: 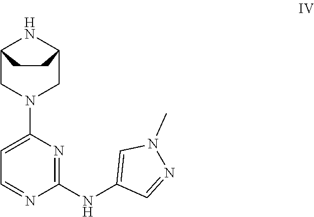 [SMILES: Cn1cc(Nc2nccc(N3C[C@H]4CC[C@@H](C3)N4)n2)cn1]
under conditions suitable to form the compound of formula I.

2. The method of claim 1, wherein R₁, R₂, R₃, and R₄ are hydrogen.

3. The method of claim 1, wherein R is p-cyanophenyl or isoquinolin-3-yl.

4. A method for preparing ap-toluenesulfonic acid salt of a compound of formula I: 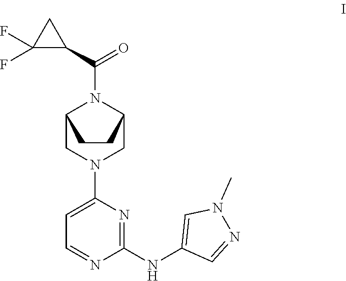 [SMILES: Cn1cc(Nc2nccc(N3C[C@H]4CC[C@@H](C3)N4C(=O)[C@@H]3CC3(F)F)n2)cn1]
comprising
(a) (i) preparing a salt of formula (II) having the structure: 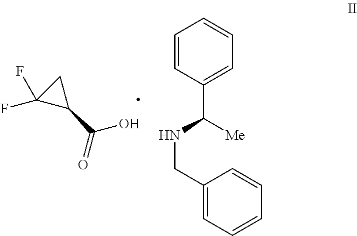 [SMILES: O=C(O)[C@@H]1CC1(F)F ; C[C@@H](NCc1ccccc1)c1ccccc1]
from (A) a compound having the structure: 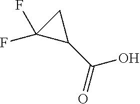
and (B) a base having the structure: 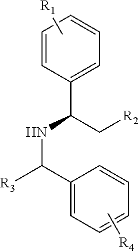
wherein R₁, R₂, R₃, and R₄ are each independently selected from the group consisting of hydrogen, halo, hydroxy, C₁-C₆ alkyl and C₁-C₆ alkoxy,
and isolating from said salt of formula II a chiral compound having the structure: 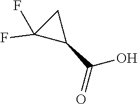
or,
(ii) preparing an activated ester having the structure: 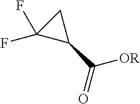
wherein R is selected from the group consisting of C₆-C₁₂ aryl and C₄-C₉ heteroaryl, wherein said C₆-C₁₂ aryl and C₄-C₉ heteroaryl are optionally substituted with a cyano, —S(═O)—R₀, —S(═O)₂—R₀, nitro, C₁-C₆ alkoxy, or halo, where R₀ is C₁-C₆ alkyl;
(b) reacting said activated ester or said chiral compound with a compound of formula IV: 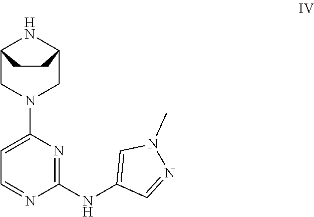 [SMILES: Cn1cc(Nc2nccc(N3C[C@H]4CC[C@@H](C3)N4)n2)cn1]
under suitable conditions to form the compound of formula I: 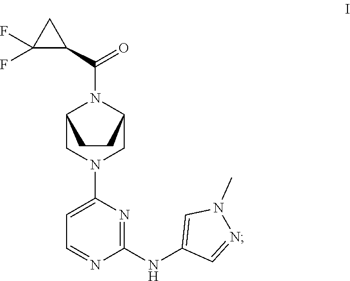 [SMILES: Cn1cc(Nc2nccc(N3C[C@H]4CC[C@@H](C3)N4C(=O)[C@@H]3CC3(F)F)n2)cn1]
and,
(c) treating said compound of formula (I) with p-toluenesulfonic acid under suitable conditions to afford the p-toluenesulfonic acid salt of the formula IA:

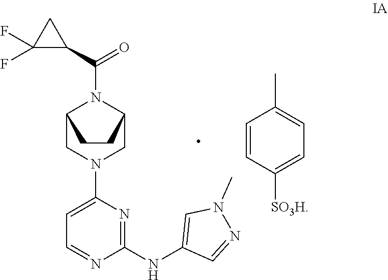

5. The method of claim 4, wherein R₁, R₂, R₃, and R₄ are hydrogen.

6. The method of claim 4, wherein R is p-cyanophenyl or isoquinolin-3-yl.

7. A method for preparing a salt of formula II: 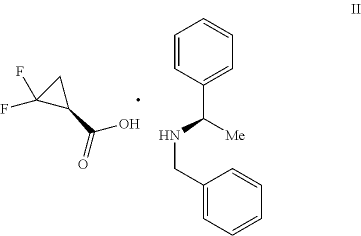 [SMILES: O=C(O)[C@@H]1CC1(F)F ; C[C@@H](NCc1ccccc1)c1ccccc1]
comprising
(a) preparing a carboxylic acid having the structure: 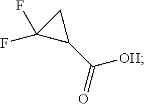
and,
(b) reacting said carboxylic acid with a compound having the structure: 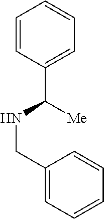 [SMILES: C[C@@H](NCc1ccccc1)c1ccccc1]
under suitable conditions to form the salt of formula II.

8. The method of claim 7, wherein the carboxylic acid is prepared by treating a compound having the structure: 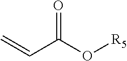
wherein R₅ is C₂-C₆ alkyl, C₃-C₆ cycloalkyl, benzyl, C₆-C₁₂ aryl, or C₄-C₉ heteroaryl with an ester compound having the structure: 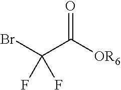
wherein R₆ is C₁-C₅ alkyl, benzyl, C₆-C₁₂ aryl, or C₄-C₉ heteroaryl.

9. The method of claim 8 wherein R₅ is n-propyl or n-butyl, and R₆ is methyl or ethyl.

10. The method of claim 7, wherein the reaction is carried out using n-Bu₄NBr, n-Bu₄NI or n-Bu₄NOH.

11. The method of claim 7, wherein the carboxylic acid is prepared by
(a) treating a compound having the structure: 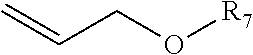
wherein R₇ is C₂-C₆ alkyl, C₃-C₆ cycloalkyl, C₆-C₁₂ aryl or C₄-C₉ heteroaryl with an ester compound having the structure: 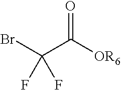
wherein R₆ is C₁-C₅ alkyl, benzyl, C₆-C₁₂ aryl, or C₄-C₉ heteroaryl under suitable conditions to form an intermediate having the structure: 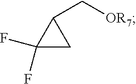
(b) treating the intermediate generated in step (a) with a Lewis acid selected from the group consisting of FeCl₃ and AlCl₃ under suitable conditions to form an alcohol compound having the structure: 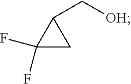
and,
(c) reacting said alcohol compound with an oxidizing agent under suitable conditions to form the carboxylic acid.

12. The method of claim 11, wherein R₇ is C₅H₁₁, and R₆ is methyl or ethyl.

13. The method of claim 11, wherein the reaction is carried out using n-Bu₄NBr, n-Bu₄NI or n-Bu₄NOH.

14. The method of claim 11, wherein the Lewis acid is FeCl₃.

15. The method of claim 11, wherein the oxidizing agent is selected from the group consisting of a periodate, a chromate, a peroxide, sodium hypochlorite and potassium hypochlorite.

16. The method of claim 11, wherein the oxidizing agent is sodium hypochlorite.

17. The method of claim 7, wherein the carboxylic acid is prepared by
(a) treating a compound having the structure: 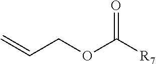
wherein R₇ is C₄-C₆ alkyl C₁-C₆ alkyl, C₃-C₆ cycloalkyl, benzyl, C₆-C₁₂ aryl, or C₄-C₉ heteroaryl with an ester compound having the structure: 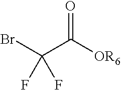
wherein R₆ is selected from the group consisting of C₁-C₅ alkyl, benzyl, C₆-C₁₂ aryl, and C₄-C₉ heteroaryl under suitable conditions to form an intermediate having the structure: 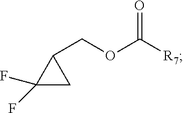
(b) treating the intermediate generated in step (a) with a base selected from the group consisting of sodium hydroxide, potassium hydroxide, lithium hydroxide, calcium hydroxide, cesium carbonate, lithium carbonate, potassium carbonate, ammonium sodium carbonate, ammonium carbonate, lithium bicarbonate, sodium bicarbonate, potassium carbonate, metal or ammonium carboxylates, mono-, and di- and tri-basic metal phosphates, under suitable conditions to form an alcoholic compound having the structure: 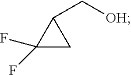
(c) reacting said alcoholic compound with an oxidizing agent selected from the group consisting of periodate, chromate, peroxide, sodium hypochlorite and potassium hypochlorite under suitable conditions to provide the carboxylic acid.

18. The method of claim 7, wherein R₇ is C₅H₁₁, and R₆ is methyl or ethyl.

19. The method of claim 7, wherein the reaction is carried out using n-Bu₄NBr.

20. The method of claim 7, wherein the base is potassium hydroxide.

21. The method of claim 7, wherein the oxidizing agent is selected from the group consisting of a periodate, a chromate, a peroxide, sodium hypochlorite and potassium hypochlorite.

22. The method of claim 7, wherein the oxidizing agent is sodium hypochlorite.
